(19) United States
(12) Patent Application Publication
(10) Pub. No.: US 2015/0182487 A1
(43) Pub. Date: Jul. 2, 2015

(54) COMPOSITION FOR PREVENTING AND TREATING INFLAMMATORY DISEASES AND IMMUNE DISEASES, CONTAINING APO-9`-FUCOXANTHINONE AS ACTIVE INGREDIENT

(71) Applicant: JEJU NATIONAL UNIVERSITY INDUSTRY ACADEMIC COOPERATION FOUNDATION, Jeju-si (KR)

(72) Inventors: Young-Sang KOH, Jeju-si (KR); Eun-Sook YOO, Jeju-si (KR); Jin-Won HYUN, Jeju-si (KR); Hee-Kyoung KANG, Jeju-si (KR); Nam-Ho LEE, Jeju-si (KR); In-Soo SUH, Jeju-si (KR)

(21) Appl. No.: 14/643,841

(22) Filed: Mar. 10, 2015

Related U.S. Application Data
(63) Continuation-in-part of application No. PCT/KR2013/001989, filed on Mar. 12, 2013.

(30) Foreign Application Priority Data
Sep. 17, 2012 (KR) 10-2012-0102646

(51) Int. Cl. A61K 31/22; A23L 1/30; A61K 36/03

(52) U.S. Cl. CPC A61K 31/22; A61K 36/03; A23L 1/3002; A23V 2002/00

(57) ABSTRACT

The present invention relates to a composition for preventing and treating inflammatory diseases and immune diseases, containing a Sargassum muticum extract and apo-9′-fucoxanthinone as active ingredients. According to the present invention, the Sargassum muticum extract and apo-9′-fucoxanthinone show an excellent inhibiting activity for the generation of IL-12, IL-6 and TNF-α, which are inflammatory cytokines, in a macrophage and a dendritic cell due to a stimulating factor causing an inflammatory response, remarkably inhibit MAPK and AP-1 activity, and inhibit the activity of NLRP3 inflammasome which is known to be involved in the pathogenesis of various immune inflammatory diseases, and thus can be ultimately useful for the development of an agent for treating inflammatory diseases and immune diseases caused by an excessive immune inflammatory response. In addition, according to the present invention, the Sargassum muticum extract and apo-9′-fucoxanthinone are derived from an edible natural product and do not cause cytotoxicity so as to be safe in vivo, and thus can also be safely used as a material for medical supplies and a dietary supplement.

### TECHNICAL FIELD

The present invention relates to a novel use of an apo-9′-fucoxanthinone compound or Sargassum muticum extracts capable of effectively preventing and treating inflammatory diseases or immune diseases.

### BACKGROUND ART

Inflammatory disorders are one of the most important health problems in the world. Inflammation is in general a localized protective response of body tissues to invasion of a host by foreign materials or injurious stimuli. Causes of inflammation can be infectious agents such as bacteria, viruses, and parasites; burns or physical agents such as radiation; chemicals such as toxins, drugs or industrial agents; immunological responses such as allergies and autoimmune responses; or conditions associated with oxidative stress.

Inflammation is characterized by pain, redness, swelling, heat, and eventual loss of function of an affected area. These symptoms are results of a series of complex interactions occurring between cells of an immune system. A response of the cells results in an interacting network of several groups of inflammatory mediators: proteins (for example, a cytokine, an enzyme (for example, protease, peroxidase), major basic proteins, adhesion molecules (ICAM, VCAM), lipid mediators (for example, eicosanoid, prostaglandin, leukotriene, a platelet activating factor (PAF)), and reactive oxygen species (for example, hydrogen peroxide, superoxide anion (O²⁻), nitric oxide (NO), and the like). However, many of those mediators of inflammation are also regulators of normal cellular activities. Therefore, deficiencies of inflammatory responses lead to an uncontrolled and compromised host (i.e. infection), and, thus, chronic inflammation leads to inflammatory diseases mediated in part by excessive production of several of the above-mentioned mediators.

In particular, autoimmune diseases as one of inflammatory diseases are characterized by a spontaneous response to an attack of an immune system of an individual against its own organs. Such a response is caused by recognition of an auto-antigen by T lymphocytes, which results in a humoral immune response (production of an autoantibody) and a cellular immune response (increase in cytotoxic activity of a lymphocyte and a macrophage). The autoimmune diseases may include: rheumatic diseases, psoriasis, systemic dermatomyositis, multiple sclerosis, lupus erythematosus, or deterioration of immune responses to antigens, i.e. asthma and allergies to drugs or foods. All of these diseases are restrictive and chronic diseases and can be fatal diseases in certain circumstances. Until the present, it has not been suggested for any effective treatment method for the above-mentioned diseases. Therefore, a drug, medicine, or a medium capable of alleviating or relieving a progressive disease can be an important means for solving a health problem of a patient. There has been made concentrated effort to find appropriate drugs and methods by searching treatment methods for autoimmune diseases. Today, treatments for autoimmune diseases are mainly based on a use of immunosuppressive drugs such as glucocorticoids, calcineurin inhibitors, and antiproliferatives-antimetabolites. However, such pharmacological treatments act on various targets and thus may deteriorate overall immune function. Otherwise, if these pharmacological treatments are used for a long time, various cytotoxic activities may suppress an immune system in a non-specific manner, so that a patient may be exposed to a risk of infectious diseases or cancers. The calcineurin and glucocorticoid may have other problems caused by their nephrotoxicity and diabetogenic properties, and, thus, they are limited in use for some clinical symptoms (for example, renal insufficiency, diabetes, and the like).

For this reason, patients with immune diseases including autoimmune disease or inflammatory diseases have special interests in treatment considered as “natural” treatment with mild anti-inflammatory effects and without major side effects, which can be used for disease prevention and as adjuvant treatment, and a lot of researchers have increased interests in development of nature-originated medicines.

Recently, there have been made some researches on natural substances to develop stable medicines with fewer side effects. As conventional technologies, Korean Patent No. 668,067 describes that phenylbutenoid derivatives isolated from ginger has excellent anti-inflammatory activity and Korean Patent No. 396,526 describes that xanthorrhizol isolated from Curcuma xanthorrhiza Roxb. has an anti-inflammatory activity and thus can be used for treating inflammatory diseases.

Further, the recent studies exhibit that bioactive substances included in seaweed increase anti-inflammatory and anti-cancer effects (Hwang pa et al. 2011, Khan M N et al. 2008). Particularly, Sargassum muticum among seaweed is known to relieve skin irritation, but few studies about other pharmacological uses of Sargassum muticum and main ingredients included in Sargassum muticum have been conducted.

Meanwhile, the inventors of the present invention have completed the present invention by finding that Sargassum muticum extracts and an apo-9′-fucoxanthinone compound isolated from the extracts have an activity of inhibiting production of inflammatory mediators such as TNF-α, IL (interleukin)-6, and IL-12 p40 in bone marrow-derived dendritic cells and macrophage stimulated with lipopolysaccharides (LPS) or CpG-oligodeoxynucleotide (CpG-ODN), an activity of inhibiting inflammation through a mechanism for inhibiting an activity of extracellular signal-regulated kinase (ERK), a member of the MAPK family and inhibiting a transcriptional activity of a transcription factor AP-1, and inhibit an activity of NLRP3 (The nucleotide-binding oligomerization domain (NOD)-like receptor family, pyrin domain containing 3) inflammasome known to be involved in the mechanism of inflammatory diseases and apoptosis of macrophage so as to be used as a therapeutic agent for preventing and treating inflammatory diseases and immune diseases.

### DISCLOSURE

### Technical Problem

An object of the present invention is to provide a pharmaceutical composition comprising apo-9′-fucoxanthinone as an effective component for preventing and treating inflammatory diseases or immune diseases.

Further, another object of the present invention is to provide a functional food comprising apo-9′-fucoxanthinone or Sargassum muticum extracts as an effective component for preventing and treating inflammatory diseases or immune diseases.

Furthermore, still another object of the present invention is to provide a pharmaceutical composition comprising Sargassum muticum extracts as an effective component for preventing and treating inflammatory diseases or immune diseases.

### Technical Solution

In order to achieve the above-described objects of the present invention, there is provided a pharmaceutical composition comprising apo-9′-fucoxanthinone as an effective component for preventing and treating inflammatory diseases or immune diseases.

According to an exemplary embodiment of the present invention, the apo-9′-fucoxanthinone may be isolated from Sargassum muticum extracts.

According to an exemplary embodiment of the present invention, the apo-9′-fucoxanthinone may have an inhibitory effect on production of pro-inflammatory cytokines stimulated with LPS (lipopolysaccharide) or CpG-ODN.

According to an exemplary embodiment of the present invention, the pro-inflammatory cytokines may be IL-12, IL-6, or TNF-α.

According to an exemplary embodiment of the present invention, the apo-9′-fucoxanthinone may inhibit activities of MAPK (MAP Kinase) and AP-1.

According to an exemplary embodiment of the present invention, the apo-9′-fucoxanthinone may have an activity of inhibiting NLRP3 (the nucleotide-binding oligomerization domain (NOD)-like receptor family, pyrin domain containing 3) inflammasome.

According to an exemplary embodiment of the present invention, the Sargassum muticum extracts may be extracted with an extractant selected from the group consisting of water, C1 to C4 alcohol, hexane, ethylacetate, butylene glycol, propylene glycol, glycerin, ether, chloroform, methylene chloride, n-butanol, and mixed solvents thereof.

According to an exemplary embodiment of the present invention, the apo-9′-fucoxanthinone may be included at a concentration of 5 to 50 μM in the composition.

According to an exemplary embodiment of the present invention, the inflammatory diseases may include sepsis exhibiting a systemic inflammatory response caused by infection with microorganisms or endotoxic shock.

According to an exemplary embodiment of the present invention, the immune diseases are autoimmune diseases caused by hyper-inflammation and may include one or more diseases selected from the group consisting of alopecia greata, dermatitis, allergy, atopy, asthma, conjunctivitis, periodontitis, rhinitis, tympanitis, pharyngolaryngitis, tonsillitis, pneumonia, gastric ulcer, gastritis, Crohn's disease, colitis, hemorrhoids, ankylosing spondylitis, lupus, fibromyalgia, psoriasis, arthritis, ostarthritis, rheumatoid arthritis, shoulder joint diseases, tendinitis, tenosynovitis, peritendinitis, myositis, hepatitis, cystitis, nephritis, sjogren's syndrome, multiple sclerosis, inflammatory bowel disease, type 1 diabetes, scleroderma, gout, neurodegenerative diseases, type 2 diabetes, silicosis, atherosclerosis, autoimmune inflammatory diseases, chronic kidney diseases and genetically inherited periodic fever syndromes, CAPS (Cryopyrin-associated periodic syndrome), FACS (familial cold autoinflammatory syndrome), MWS (Muckle-Wells syndrome), NOMID (neonatal onset multisystem inflammatory disease), and acute and chronic inflammatory diseases.

Further, the present invention provides a functional food comprising apo-9′-fucoxanthinone or Sargassum muticum extracts as an effective component for preventing and treating inflammatory diseases or immune diseases.

Furthermore, the present invention provides a pharmaceutical composition comprising Sargassum muticum extracts as an effective component for preventing and treating inflammatory diseases or immune diseases.

According to an exemplary embodiment of the present invention, the Sargassum muticum extracts may be extracted with an extractant selected from the group consisting of water, C1 to C4 alcohol, hexane, ethylacetate, butylene glycol, propylene glycol, glycerin, ether, chloroform, methylene chloride, n-butanol, and mixed solvents thereof.

According to an exemplary embodiment of the present invention, the Sargassum muticum extracts may be included at a concentration of 5 to 50 μg/ml in the composition.

According to an exemplary embodiment of the present invention, the Sargassum muticum extracts may include an apo-9′-fucoxanthinone compound.

### Advantageous Effects

According to the present invention, Sargassum muticum extracts and an apo-9′-fucoxanthinone compound have an excellent activity of inhibiting production of proinflammatory cytokines such as IL-12, IL-6, and TNF-α in macrophage and dendritic cells stimulated with a inflammatory stimuli, an excellent effect of inhibiting activity of MAPK and AP-1, and inhibit an activity of NLRP3 inflammasome known to be involved in the mechanism of various inflammatory diseases and apoptosis of macrophage. Thus, it can be ultimately used for development of medicines for treating inflammatory diseases and immune diseases which can be caused by hyperactive inflammatory responses. Further, the Sargassum muticum extracts and the apo-9′-fucoxanthinone are derived from an edible natural substance and do not cause cellular toxicity and are stable in the body. Thus, they can be safely used as materials for drugs and functional foods.

### DESCRIPTION OF DRAWINGS

FIG. 1 illustrates a structural formula of an apo-9′-fucoxanthinone compound according to the present invention.

FIGS. 2A-AB illustrate a result of ELISA assay on an inhibitory effect of Sargassum muticum extracts of the present invention on production of proinflammatory cytokines IL-12 p40, IL-6, and TNF-α in bone marrow-derived macrophage (2A) and dendritic cells (2B) stimulated with CpG-ODN, and ND represents non-detected and SME represents Sargassum muticum extracts according to the present invention.

FIGS. 3A-3B illustrate a result of ELISA assay on an inhibitory effect of Sargassum muticum extracts according to the present invention on production of proinflammatory cytokines IL-12 p40, IL-6, and TNF-α in bone marrow-derived macrophage (3A) and dendritic cells (3B) stimulated with LPS.

FIG. 4 illustrates a result of ELISA assay on an inhibitory effect of apo-9′-fucoxanthinone compound of the present invention on production of proinflammatory cytokines IL-12 p40, IL-6, and TNF-α in bone marrow-derived macrophage stimulated with CpG-ODN, and ND represents non-detected and APO-9′ represents apo-9′-fucoxanthinone compound according to the present invention.

FIG. 5 illustrates a result of ELISA assay on an inhibitory effect of apo-9′-fucoxanthinone compound according to the present invention on production of proinflammatory cytokines IL-12 p40, IL-6, and TNF-α in dendritic cells stimulated with CpG-ODN.

FIGS. 6A-6B illustrate a result of measurement on level of phosphorylation (6A) of ERK, JNK, and p38 and level of degradation and phosphorylation (6B) of IκBα by the western blot in order to examine inhibitory effect of an apo-9-fucoxanthinone compound according to the present invention on MAPK and NF-κB activity.

FIG. 7 is a graph exhibiting a result of luciferase assay of HEK293T cells transfected with TLR9-expression plasmid and treated at different concentrations of apo-9′-fucoxanthinone compound and then stimulated with CpG-ODN in order to analyze inhibitory effect on AP-1 reporter activity of the apo-9′-fucoxanthinone compound according to the present invention.

FIG. 8 is a graph exhibiting a result of luciferase assay of HEK293T cells transfected with TLR9-expression plasmid and treated at different concentrations of apo-9′-fucoxanthinone compound and then stimulated with CpG-ODN in order to analyze inhibitory effect on NF-κB reporter activity of apo-9′-fucoxanthinone compound according to the present invention.

FIG. 9 illustrates a result of MTT analysis on a degree of apoptosis caused by apo-9′-fucoxanthinone compound according to the present invention and ELISA on a change in production of IL-1β in bone marrow-derived macrophage stimulated with LPS and treated with each of the apo-9′-fucoxanthinone compound or parthenolide and then treated with ATP in order to confirm whether or not apo-9′-fucoxanthinone compound has an inhibitory effect of apoptosis of macrophage and activity of NLRP3 inflammasome, and ND represents non-detected, Part represents parthenolide, and APO-9′ represents the apo-9′-fucoxanthinone compound according to the present invention.

### BEST MODE OF THE INVENTION

The present invention is characterized by providing a novel use of an apo-9′-fucoxanthinone compound as a new medicine isolated from a natural substance for treating inflammatory or immune diseases with excellent anti-inflammatory activity without causing cellular toxicity.

To be specific, according to the present invention, the apo-9′-fucoxanthinone compound may be a compound isolated from Sargassum muticum extracts and having a structural formula represented by the following Chemical Formula.

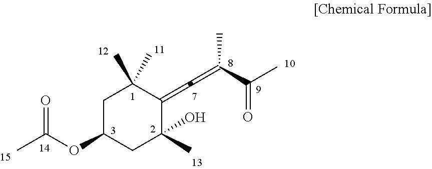

The apo-9′-fucoxanthinone compound according to the present invention can be separated from a natural substance or can be produced by a chemical synthesis method publicly known in the art. Preferably, the apo-9′-fucoxanthinone compound can be isolated and purified from solvent extracts obtained from Sargassum muticum.

Preferably, Sargassum muticum is extracted with methanol as a solvent and additionally fractionated into n-hexane, methylene chloride, ethyl acetate, n-butanol, and water fractions. Then, an apo-9′-fucoxanthinone compound can be isolated and purified by conducting chromatography to the methylene chloride fractions.

Further, the Sargassum muticum may employ Sargassum muticum being sold in the market, and preferably, sufficiently dried Sargassum muticum may be used before being extracted with a solvent. In a preferable example, dried Sargassum muticum is immersed in alcohol, preferably, methanol and then stirred and filtered so as to obtain extracts. Then, the obtained extracts undergo a concentration process, and the concentrated extracts are suspended in water. Then, various organic solvents are used to obtain fractions.

Furthermore, in the chromatography to be conducted to the obtained fractions, to isolate main active substances included in the fractions, various column chromatography such as silica gel column chromatography, HPLC (ThermoFisher Scientific, USA), and the like may be conducted alone or in parallel with each other so as to isolate and purify the compound of the present invention.

Meanwhile, the Sargassum muticum extracts and the apo-9′-fucoxanthinone compound of the present invention obtained by the above-described process can effectively inhibit production of proinflammatory cytokines which may cause inflammatory diseases and immune diseases.

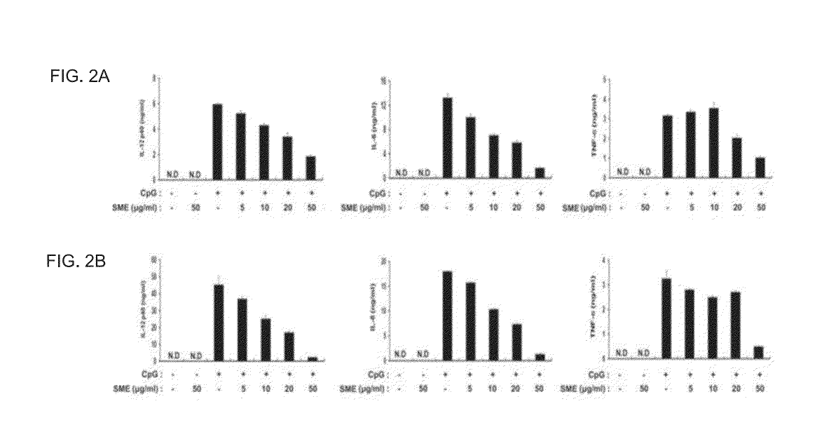

That is, according to an exemplary embodiment of the present invention, it was observed that when bone marrow-derived macrophage and dendritic cells were treated with each of the Sargassum muticum extracts or the apo-9′-fucoxanthinone compound of the present invention, production of proinflammatory cytokines (for example, IL-12, IL-6, TNF-α) stimulated with LPS or CpG-ODN can be effectively inhibited (refer to FIG. 2 to FIG. 5).

For reference, lipopolysaccharide (LPS) as an endotoxic substance induces production of an inflammation-inducing factor in dendritic cells and promotes production of pro-inflammatory cytokines that cause inflammatory responses. That is, when external stimuli that may cause inflammatory responses are applied, expression of proinflammatory cytokines such as TNF-α, and the like is induced. The produced proinflammatory cytokines stimulate expression of genes for coding iNOS and COX-2, and NO and PGE-2, that are involved in inflammatory responses are produced so as to cause inflammatory responses.

Therefore, when inflammation-inducing substances for the proinflammatory cytokines such as TNF-α, IL-6, or IL-12 are excessively secreted or the cells are kept in an activated state for a long time, a serious side effect such as tissue damage can be caused.

Further, a toll-like receptor (TLR) is mainly expressed in immune cells and responsible for an important function for immunological activity.

It is known that TLRs detect PAMPs (pathogen-associated molecular patterns) and stimulate immune cells via the MyD88-dependent TLR signaling pathway, which leads to activation of the MAPK signaling pathway, the transcription factors NF-κB and AP-1. Ten functional family members of TLRs (TLR1 to TLR10) have been identified in humans [Akira S. et al., Nature Immunol., 2, 675 to 680 (2001)]. TLR2, TLR4, and TLR5 are crucial for the recognition of peptidoglycan, lipopolysaccharide, and flagellin, respectively, and TLR6 associates with TLR2 and recognizes lipoproteins from mycoplasma [Ozinsky, A., et al., Proc. Natl. Acad. Sci USA., 97, 13766-13771 (2000)]. It is known that TLR9 detects unmethylated CpG motifs present in bacterial or viral DNA, and TLR3 activates immune cells in response to double-stranded RNA [Hemmi, H. et al., Nature, 408, 740-745 (2000)].

In particular, TLR9 is a type of pattern recognition receptor located in the front line in a cell and recognizes specific sites of various intruding pathogens and sends a signal into the cell so as to induce an immune response. Unlike the other TLRs expressed on the cell membrane, TLR9 is known to be expressed in the endosome in the cell and recognize CpG motifs in short viral or bacterial DNA sequences. It is known that if a TLR9-expressing immune cell or a specific cancer cell is treated with ligands, MyD88 transmits a signal into the cell so as to activate NF-κB and AP-1, thereby expressing various proinflammatory cytokines such as IL-6, IL-8, IL-12, TNF-α, and the like and type 1 interferon.

Meanwhile, the apo-9′-fucoxanthinone compound of the present invention can inhibit production of inflammatory mediators such as TNF-α, IL-6, or IL-12, in dendritic cells and macrophage stimulated with lipopolysaccharides (LPS, TLR4 ligand) or CpG-ODN (TLR9 ligand).

Herein, the interleukin-12 (IL-12) is a type of interleukins produced in antigen-presenting cells such as dendritic cells, macrophages, and the like by PAMP (pathogen-associated molecular pattern) stimuli, and stimulates and differentiates T cells into type 1 helper T cells and plays an important role in activities of NK (natural killer) cells and T lymphocytes. This is a heterodimer composed of IL-12A (p35) and IL-12B (p40). Since expression of the IL-12A (p35) is constitutive, the biological function of the interleukin-12 depends mainly on expression of the IL-12B (p40). There has been reported a study exhibiting that such inhibition of interleukin-12 is helpful to treat autoimmune diseases, particularly type 1 helper T cell-mediated autoimmune diseases.

Therefore, the apo-9′-fucoxanthinone compound of the present invention can be used for treating and preventing inflammatory diseases with an activity of inhibiting production of TNF-α, IL-6, and IL-12.

In particular, the “inflammatory diseases” in the present invention are caused by overproduction of proinflammatory cytokines TNF-α, IL-6, or IL-12 induced by stimuli of lipopolysaccharide (LPS) or CpG-ODN, and may include sepsis or autoimmune diseases.

To be more specific, the autoimmune diseases may include alopecia greata, dermatitis, allergy, atopy, asthma, conjunctivitis, periodontitis, rhinitis, tympanitis, pharyngolaryngitis, tonsillitis, pneumonia, gastric ulcer, gastritis, Crohn's disease, colitis, hemorrhoids, gout, ankylosing spondylitis, rheumatic fever, lupus, fibromyalgia, psoriasis, arthritis, ostarthritis, rheumatoid arthritis, shoulder joint diseases, tendinitis, tenosynovitis, peritendinitis, myositis, hepatitis, cystitis, nephritis, sjogren's syndrome, multiple sclerosis, and acute and chronic inflammatory diseases, and the sepsis may include systemic inflammatory response syndrome (SIRS) caused by infection with microorganisms and exhibiting symptoms, such as fever characterized by an increase in body temperature to be 38° C. or more, hypothermia characterized by an increase in body temperature to be 36° C. or less, an increase in breathing rate to be 24 times per minute (tachypnea), a heart rate of 90 times per minute (tachycardia), an increase or a remarkable decrease in number of white blood cells according to the blood test result, and endotoxic shock.

More preferably, the autoimmune diseases may include: (1) rheumatoid arthritis, caused when the immune system attacks various articular tissues; (2) multiple sclerosis (MS) which is an autoimmune disease of the central nervous system caused by T cells and allows persons with MS to live relatively normally in most cases but may cause blindness, paralysis, or premature death in more severe cases; (3) immune-mediated or type I diabetes mellitus, in which immune cells destroy insulin-producing cells in the pancreas and for which the MHC plays an important role; (4) inflammatory bowel diseases, caused when the immune system attacks the intestine; (5) scleroderma, which induces the thickening of the skin or vessels; (6) systemic lupus erythematosus (SLE), a systemic autoimmune disease that is accompanied by severe fatigue, rash, joint pains, and the like and can harm the kidneys, the brain, the lungs, and the like in more severe cases. Further, the sepsis may include systemic inflammatory response syndrome (SIRS) caused by infection with microorganisms and exhibiting inflammatory response all over the body and septic (endotoxic) shock. In particular, the endotoxic shock (septic shock) is caused mainly by overproduced interleukin-6 and TNF-α.

Therefore, the apo-9′-fucoxanthinone compound and the Sargassum muticum extracts of the present invention can be used as a pharmaceutical composition for preventing and treating inflammatory diseases, particularly, sepsis, and immune diseases, particularly, autoimmune disease.

Further, the inventors of the present invention established that inhibition of production of proinflammatory cytokines by the apo-9′-fucoxanthinone compound and the Sargassum muticum extracts according to the present invention was ascribed to inhibition of an activity of MAPK as their superordinate signaling system. If PRRs (pattern recognition receptors) recognize PAMPs and bind to the PAMPs, signal transduction pathways for NF-κB, MAPK and AP-1 are activated, thereby producing inflammation-inducing bioactive substances. Therefore, if signal transduction for NF-κB, AP-1 and MAPK is inhibited, production of inflammation-inducing bioactive substances can be inhibited. Eventually, inflammatory and immune diseases can be prevented.

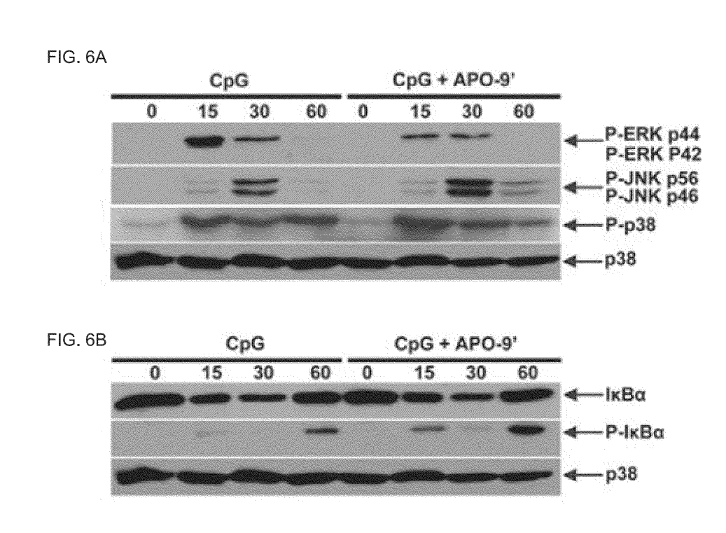

In order to confirm this matter, in an exemplary embodiment of the present invention, a macrophage treated with the apo-9′-fucoxanthinone compound according to the present invention and a non-treated macrophage were treated with CpG-ODN (TLR9 ligand) to cause inflammation. Then, an effect on phosphorylation of MAPK and degradation and phosphorylation of IκBα was analyzed. As a result, it was exhibited that the group treated with the apo-9′-fucoxanthinone compound suppresses signaling activity by suppressing phosphorylation of MAPK (i.e. phosphorylation of ERK (the extracellular signal-activated kinases) MAPK) as compared with the control group which was not treated with the compound (refer to FIG. 6A). On the other hand, it was exhibited that the compound of the present invention does not have an effect on phosphorylation of IκBα. That is, it was exhibited that in the bone marrow-derived macrophage stimulated with CpG-ODN, phosphorylation and degradation of IκBα occurred within 15 minutes after stimulation, but the group treated with the compound of the present invention did not exhibit any particular difference from the group which was not treated with the compound (refer to FIG. 6B). Accordingly, based on this result, the inventors of the present invention found that the compound of the present invention can specifically inhibit an activity of MAPK in response to stimuli of CpG-ODN.

For reference, MAPK is well known as a representative signal transduction pathway through which an extracellular stimulus is transmitted from a cell membrane to a nucleus. The MAPK transmits activated signals from a growth hormone, a cytokine, and a stress into a cell, and plays various roles for proliferation, differentiation, and apoptosis of a cell. The MAPK can be roughly classified into 1) an extracellular signal-activated kinase (ERK), 2) a c-JUN N-terminal kinase (JNK), and 3) p38 MAPK. The ERK (ERK1/2) is mainly involved in signal transduction of a growth hormone and plays a key role in proliferation and differentiation of a cell. Meanwhile, it is known that the p38 MAPK and the JNK classified as stress kinases are activated by extracellular stress stimuli and mediate in inflammatory responses, immune responses, and cell apoptosis. Therefore, it is possible to treat inflammatory and immune diseases by using these MAP kinase inhibitors.

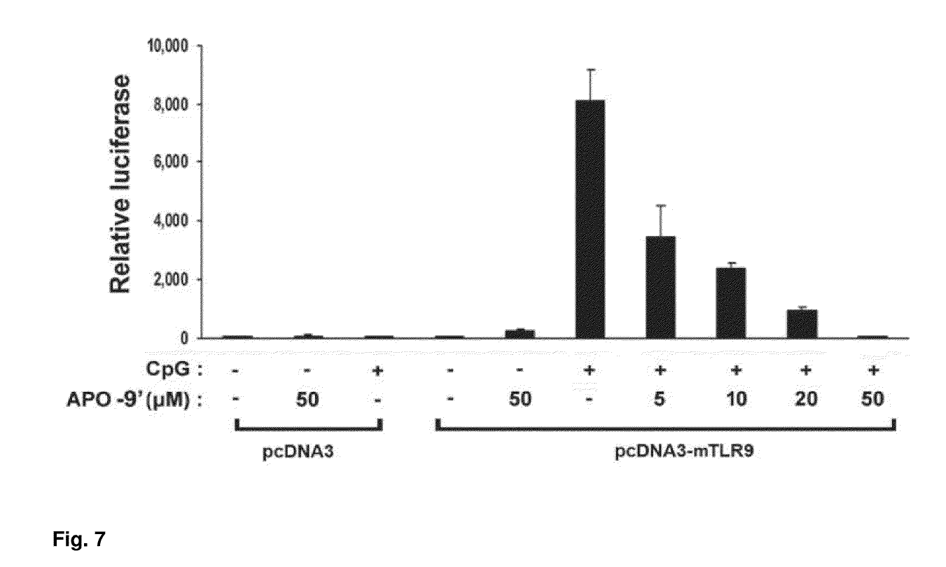

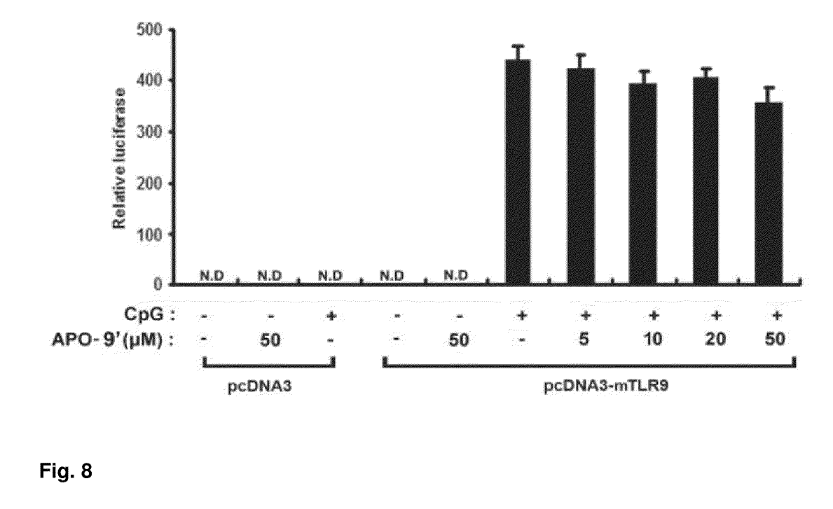

Further, the inventors of the present invention examined an effect of the apo-9′-fucoxanthinone compound on a reporter activity of NF-κB and AP-1 in order to confirm whether the apo-9′-fucoxanthinone compound has an activity of inhibiting gene expression of these cytokines in production of pro-inflammatory cytokines. As a result, it was exhibited that the apo-9′-fucoxanthinone compound cannot inhibit a transcriptional activity of TLR9-dependent NF-κB stimulated with CpG-ODN but can inhibit a transcriptional activity of TLR9-dependent AP-1 in a dose-dependent manner (refer to FIG. 7 and FIG. 8).

Further, the apo-9′-fucoxanthinone compound of the present invention has an activity of inhibiting an activity of NLRP3 inflammasome.

When a human body is infected by pathogens, an innate immune response is activated as a primary defense mechanism. In this case, as the first response, PRRs (pattern recognition receptors) present in the host cell recognize a PAMP (pathogen-associated molecular pattern) of the invading pathogen. Further, a signal through the PRRs activates various inflammatory pathways, that is, MAPK, caspase-1, NF-κB, AP-1 (activator protein-1), and the like and induces an inflammatory response.

Representative examples of the PRRs may include a TLR (Toll-like receptor) positioned on a membrane surface and an NLR (nucleotide-binding oligomerization domain (NOD)-like receptor) present within the cytoplasm.

Further, the inflammasome refers to a protein complex that recognizes a molecular pattern involved in stress or cell damage in the cytoplasm, and it is a complex that includes one of various NLR (nucleotide-binding oligomerization domain (NOD)-like receptor) proteins and activates a signal transduction pathway for caspase-1, resulting in activating pro-inflammatory cytokines IL-1β and IL-18 to be secreted. Among the activated pro-inflammatory cytokines, IL-1β serves as a potential endogenous pyrogen and induces flu-like symptoms such as chills, rigors, fever, nausea, vomiting, headache and fatigue when injected into humans at 1-10 nano grams/kg of body weight. Both IL-1β and IL-1α bind to the IL-1 receptor (IL1-R) and induce the formation of a high-affinity ternary complex with the IL-1R accessory protein. The resulting downstream signaling cascade leads to transcription factor induction of proinflammatory cytokines and chemokines, and includes genes required for angiogenesis and the recruitment of immune effector cells into the extravascular space.

While these responses to IL-1β are critical for host protection from many types of viruses and microbes and may aid in cellular and tissue repair responses, the dysregulation of IL-1β activity is now implicated in a variety of seemingly divergent diseases such as type II diabetes and gout.

Further, the compound according to the present invention particularly inhibits NLRP3 inflammasome. Proteins regulating secretion of IL-1 when receiving an inflammatory signal as described above form complexes in a cell, and these complexes are referred to as “inflammasome” which is a complex of various proteins. One of the main proteins is NLRP3. NLRP3 inflammasome is an important component of the innate immune response against a broad range of microbial and self-generated danger signals, including bacterial pore forming toxins, ATP, and MSU crystals. The molecular mechanism of activation of the NLRP3 inflammasome by these diverse stimuli is still unclear, but evidence suggests that NLRP3 is activated by a two-step mechanism. In the first step, known as the priming step, NLRP3 is transcriptionally up-regulated by NF-κB-inducing stimuli such as ligands of the Toll like receptors. However, an increase in expression of NLRP3 protein is not sufficient to induce activities thereof, and an additional step is needed. The second step includes activating NLRP3 inflammasome by a danger signal such as ATP and MSU crystals and microbial stimuli such as pore-forming toxins of bacteria then leading to an activation of caspase-1, resulting in activating IL-1β. The activated caspase-1 induces cell death called “pyroptosis”.

Pyroptotic cell death is a form of programmed cell death and is caspase-1 dependent and induces inflammation. Further, pyroptosis differ from apoptosis as apoptotic cell death results in the orderly degradation and clearance of cellular contents, whereas pyroptosis, like necrosis, results in the release of cellular contents to the extracellular environment.

Further, if NLRP3 inflammasome is activated in an abnormal manner, development of various inflammatory diseases is induced. Examples of such diseases may include gout, neurodegenerative diseases, type 2 diabetes, silicosis, atherosclerosis, autoinflammatory diseases, chronic kidney diseases, and genetically inherited periodic fever syndromes. Therefore, the above-described diseases can be prevented and treated by inhibiting an activity of NLRP3 inflammasome.

Details of an effect of NLRP3 inflammasome on development of the above-described diseases will be explained. An inflammatory response plays a very important role in development of chronic kidney diseases. In this regard, it was found that inflammasome-dependent cytokines such as IL-1β and IL-18 play an important role in chronic kidney diseases (Viaysane et. al., 2010), and NLRP3 inflammasome is activated while renal injury occurs. Compared with wild-type mice, NLRP3^{-/-} mice had less tubular injury, inflammation, and fibrosis associated with a reduction in caspase-1 activation and maturation of IL-1β and IL-18. Therefore, according to this result, NLRP3 inflammasome can be a new drug target for chronic kidney diseases, and particularly, if an activity of NLRP3 inflammasome is inhibited, chronic kidney diseases can be prevented and treated.

As another example of a disease, atherosclerosis which is one of chronic inflammatory diseases is caused by accumulation of cholesterol on the innermost wall covering a blood vessel and proliferation of endothelial cells. An accumulation of cholesterol activates NLRP3 inflammasome and releases IL-113 in mouse and human macrophages (Wen et. al., 2012; Duewell et al., 2010). Further, according to the studies of researchers, monosodium urate, silica, asbestos, and alum crystals activate caspase-1 in a NLRP3 inflammsome dependent manner, and promote production of IL-1β and IL-18, thereby developing and aggravating the disease. In particular, development of the acute and chronic inflammatory responses known as gout and pseudogout are associated with the deposition of MSU (monosodium urate) or CPPD (calcium pyrophosphate dihydrate) crystals, respectively. MSU and CPPD engage caspase-1-activating NLRP3 inflammsome, resulting in the production of active interleukin (IL)-1β and IL-18 (Maranon et al., 2006).

Further, diseases such as periodic fever syndrome, CAPS (Cryopyrin-associated periodic syndrome), FACS (familial cold autoinflammatory syndrome), MWS (Muckle-Wells syndrome), NOMID (neonatal onset multisystem inflammatory disease) are also caused by excessive activation of NLRP3 due to genetic mutation and a resultant increase in production of IL-1β.

Therefore, according to the above descriptions, the inventors of the present invention confirmed that the compound of the present invention has an effect of inhibiting an activity of NLRP3 inflammsome, and found that the compound can be used as a new drug for NLRP3 inflammsome-relevant inflammatory diseases and immune diseases.

Accordingly, the present invention can provide a pharmaceutical composition comprising apo-9′-fucoxanthinone or Sargassum muticum extracts for preventing and treating inflammatory diseases or immune diseases.

Further, according to the present invention, the apo-9′-fucoxanthinone compound may be used in the form of salt and preferably in the form of pharmaceutically acceptable salt. Preferably, as the salt, there may be used an acid addition salt formed with a pharmaceutically acceptable free acid. As the free acid, an organic acid and an inorganic acid may be used. The organic acid may include, but is not limited to, an citric acid, an acetic acid, a lactic acid, a tartaric acid, a maleic acid, a fumaric acid, a formic acid, a propionic acid, an oxalic acid, a trifluoroacetic acid, a benzoic acid, a gluconic acid, a meta sulfonic acid, a glycolic acid, a succinic acid, a 4-toluenesulfonic acid, a glutamic acid, and an aspartic acid. Further, the inorganic acid may include, but is not limited to, a hydrochloric acid, a bromic acid, a sulfuric acid, and a phosphoric acid.

Furthermore, the composition according to the present invention may comprise only apo-9′-fucoxanthinone or Sargassum muticum extracts in a pharmaceutically effective amount or may comprise one or more pharmaceutically acceptable carriers, excipients, or diluents. The pharmaceutically effective amount means an amount sufficient to prevent, improve, and treat symptoms of the inflammatory or immune diseases. The apo-9′-fucoxanthinone compound may be included at a concentration of 5 to 50 μM in the composition, and the Sargassum muticum extracts may be included at a concentration of 5 to 50 μg/ml in the composition.

Moreover, according to the present invention, a pharmaceutically effective amount of the apo-9′-fucoxanthinone or Sargassum muticum extracts is 0.5 to 100 mg/day/body weight kg and preferably 0.5 to 10 mg/day/body weight kg. However, the pharmaceutically effective amount can be appropriately changed depending on a degree of symptoms of inflammatory or immune diseases, an age of a patient, a weight of a patient, a health condition of a patient, a sex of a patient, an administering route, a period of treatment, and the like.

The pharmaceutically acceptable composition means that it is physiologically acceptable and an allergic reaction or the similar effect thereof, such as a gastroenteric trouble, and dizziness, is not caused typically when being administered to humans. Examples of the carrier, excipient, and diluents may include lactose, dextrose, sucrose, sorbitol, mannitol, xylitol, erythritol, maltitol, starch, acacia rubber, alginate, gelatin, calcium phosphate, calcium silicate, cellulose, methylcellulose, polyvinylpyrrolidone, water, methyl hydroxybenzoate, propyl hydroxybenzoate, talc, magnesium stearate, and minerals. Further, they may additionally include fillers, anticoagulants, lubricants, wetting agents, flavoring, emulsifying agents, preservatives, and the like.

Further, the composition of the present invention may be formulated by using a method publicly known in the art in order to provide rapid, continuous, or delayed release of an active component after being administered to mammals. A formulated form may be powder, granule, tablet, emulsion, syrup, aerosol, soft or hard gelatin capsule, a sterile injection solution, or a sterile powder.

The composition according to the present invention can be administered in various ways for example, orally, percutaneously, subcutaneously, intravenously or intramuscularly. A dosage of the active component may be selected appropriately depending on various factors, such as an administering route, an age, a sex, and a weight of a patient, severity of a patient, and the like. Further, the composition for preventing and improving symptoms of inflammatory or immune diseases of the present invention may be administered along with a compound publicly known as having an effect of preventing, improving, or treating symptoms of inflammatory or immune diseases.

Therefore, the present invention can provide a drug comprising the composition including apo-9′-fucoxanthinone or Sargassum muticum extracts for preventing and treating symptoms of inflammatory or immune diseases.

Further, in the present invention, the above-described inflammatory diseases and immune diseases are caused by hyperactive inflammatory responses and are characterized in that the body excessively responds to non-specific stimuli due to an uncontrolled inflammatory response. Such hyperactive inflammatory responses cause pathological changes that induce development and chronic fixation of diseases. Concerning the definitions and examples of hyperactive inflammatory diseases, reference is made to EP-0 673 646.

Moreover, the apo-9′-fucoxanthinone or Sargassum muticum extracts according to the present invention does not induce toxicity to cells and does not cause side effects, and, thus, it can be used safely in the body. Accordingly, the apo-9′-fucoxanthinone or Sargassum muticum extracts can be used as a food composition for preventing and improving inflammatory or immune diseases.

Therefore, the food composition comprising the apo-9′-fucoxanthinone or Sargassum muticum extracts for preventing and improving inflammatory or immune diseases can be used easily for foods, such as main materials or added materials of foods, food additives, functional foods or beverages, effective in preventing and improving symptoms of inflammatory and immune diseases.

According to the present invention, the term “food” means a natural substance or a processed material that may include one or more nutrients and preferably to be edible after some processes. Typically, it includes all of foods, food additives, functional foods, and beverages.

The foods that can include the composition for preventing and improving symptoms of inflammatory and immune diseases according to the present invention may include, for example, all sorts of foods, beverages, gums, teas, vitamin complexes, functional foods, and the like. Additionally, the foods of the present invention may be special nutritious foods (for example, milk formulas, young children and baby foods, and the like), processed meat products, fish meat products, bean cured foods, jellied foods, noodles (for example, ramens, noodles, and the like), breads, dietary supplements, seasoning foods (for example, soy sauce, soybean paste, red pepper paste, mixed soy paste, and the like), sauces, confectionery (for example, snacks), candies, chocolates, gums, ice creams, milk products (for example, fermented milk, cheese, and the like), other processed foods, Kimchi, salt-fermented foods (all sorts of Kimchis, pickled vegetables, and the like), beverages (for example, fruit beverages, vegetable beverages, soybean milk, fermented drinks, and the like), natural seasonings (for example, ramen soup base powder, and the like), and the like, but the present invention is not limited thereto. The above-described foods, beverages, or food additives can be produced by using a typical production method.

Further, the term “functional food” refers to a food group added with added value such that a function of the food can act or can be expressed for a particular purpose by using physical, biochemical, and biotechnological methods, and the like and also a processed food designed to sufficiently express a body modulating function of the body related to biological defensive rhythm control, prevention of and recovery from diseases, and the like. To be specific, it may be a health functional food. The functional food may include sitologically acceptable food supplementary additives and may further include appropriate carriers, excipients, and diluents typically used for preparing a functional food.

Furthermore, the term “beverage” refers to a generic term for drinks for quenching thirst or enjoying taste, and may include a functional beverage. The beverage comprises, as an essential component, the composition for preventing and improving symptoms of inflammatory and immune diseases at a directed rate and may comprise other components without any particular limitation and also may include various flavoring agents, natural carbohydrates, and the like as additional components like other typical beverage.

In addition to the above-described components, the food comprising the composition for preventing and improving symptoms of inflammatory and immune diseases according to the present invention may include various nutritional supplements, vitamins, minerals (electrolyte), flavoring agents, such as synthetic flavoring agents, natural flavoring agents, and the like, colorings, fillers (cheese, chocolates, and the like), a pectic acid and salts thereof, an alginic acid and salts thereof, an organic acid, protective colloid thickeners, pH control agents, stabilizers, preservatives, glycerin, alcohol, and carbonating agents used for carbonated drinks, and the like. The above-described components may be used alone or in combination.

In the food including the composition for preventing and improving symptoms of inflammatory and immune diseases of the present invention, the composition of the present invention may be included in the amount of 0.001 wt % to 90 wt %, and preferably, 0.1 wt % to 40 wt %. As for the beverage, it may be included in the amount of 0.001 g to 2 g, and preferably 0.01 g to 1 g based on 100 ml. However, when the composition is taken for a long time for improving health and hygiene or for managing health, it may be included in the amount equal to or less than the above ranges. Since an effective component has no problem in terms of safety, it may be used in the amount equal to or greater than the above ranges. Therefore, the present invention is not limited thereto.

Hereinafter, the present invention will be described in more detail with reference to Examples. However, Examples will be provided only for illustrating the present invention. Thus, it would be obvious to those skilled in the art that the scope of the present invention is not limited to Examples.

### MODES OF THE INVENTION

### Example 1

### Preparation of Sargassum muticum Extracts and Isolation of Apo-9′-Fucoxanthinone Compound

<1-1> Preparation of Sargassum muticum Extracts

As a material for experiments, brown algae Sargassum muticum was collected on Jeju Island, and a voucher specimen has been deposited at the herbarium of Jeju Biodiversity Research Institute (JBRI) and given a deposit number of JBRI-10067. Sargassum muticum extracts were obtained by drying the Sargassum muticum collected on Jeju Island in the shade, extracting the dried Sargassum muticum (100 g) with 80% ethanol (2 L) three times at room temperature for 24 hours, filtering the resultant product, and then vacuum-evaporating the resultant product with a rotary evaporator. As a result, ethanol-soluble fraction of the extracts of the Sargassum muticum were obtained.

<1-2> Isolation of Apo-9′-Fucoxanthinone Compound

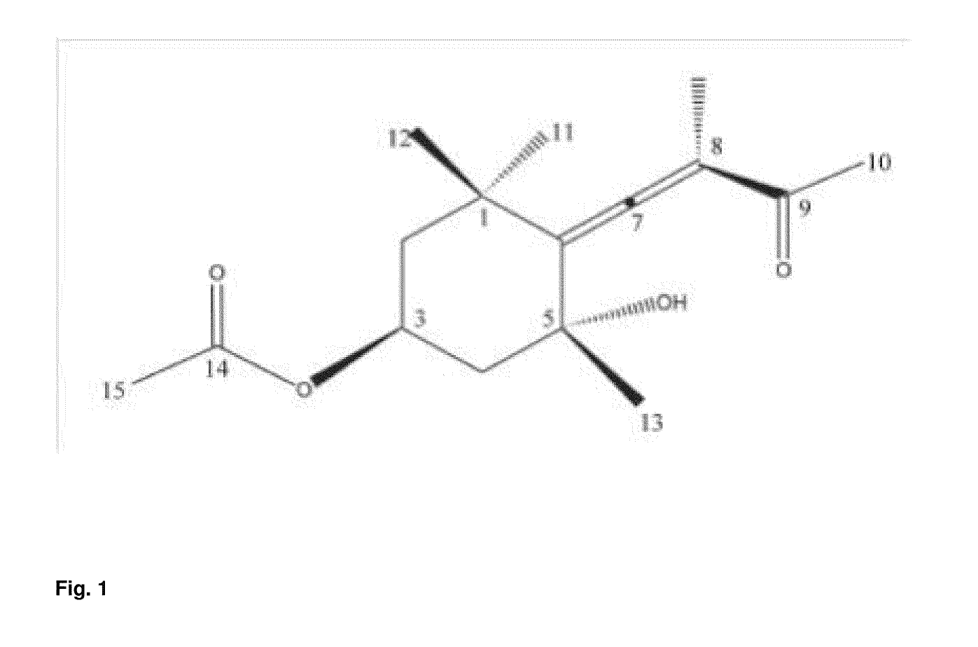

A process of immersing 2 kg of the dried brown algae Sargassum muticum in an 80% aqueous methanol solution at room temperature with stirring for two days and filtering the resultant product was repeated three times. Then, the filtrate was vacuum-dried and suspended in 1 L of distilled water and then fractionated into n-hexane, methylene chloride, ethyl acetate, n-butanol, and water fractions. From among these fractions, 5.4 g of the methylene chloride fractions were adsorbed onto a celite surface and eluted with 500 mL each and then fractionated (hexane/CH₂Cl₂ 1:0, 10:1, 5:1, 2:1, 0:1, CH₂Cl₂, ethyl acetate, methanol were used as an eluent). Then, CH₂Cl₂ fraction from the celite fraction was subjected to silica gel column chromatography (3 cm×70 cm, hexane/ethyl acetate/methanol 2:1:0.1), and, thus, 1.8 mg of apo-9′-fucoxanthinone was obtained. A structure of the isolated compound was analyzed, and a structural formula of the compound was as illustrated in FIG. 1.

### Example 2

### Analysis of Inhibitory Activity of Sargassum muticum Extracts and Apo-9′-Fucoxanthinone Compound on Pro-Inflammatory Cytokine Production

In order to check whether the Sargassum muticum extracts and apo-9′-fucoxanthinone compound isolated in Example 1 has an anti-inflammatory and an immunomodulatory activity, the following experiments were conducted. Cells and specimens used for the experiment were as described below.

Preparation of Mice

C57BL/6 mice purchased from Orient Bio Inc. were used as mice, and raised in a specific pathogen-free condition in compliance with the guidelines of National Institutes of Health. Further, all the experiments using the mice were conducted in compliance with the regulations of Jeju University Institutional Animal Care and Use Committee (#2010-0028).

Preparation of Mouse Bone Marrow-Derived Macrophage and Dendritic Cells

Bone marrow-derived dendritic cells (BMDC) and macrophage (BMDM) were obtained from a wild-type C57BL/6 mouse. To be brief, bone marrow cells were obtained by flushing a tibia and femur of the mouse with DMEM (Dulbecco's modified Eagles medium). The cells were cultured in a medium RPMI 1640 including 10% heat-inactivated FBS (Gibco, NY, U.S.A.), 50 μM of β-mercapto ethanol, and 2 mM glutamine and supplemented with 3% hybridoma cell culture supernatant J558L containing granulocyte-macrophage colony-stimulating factor (GM-CSF) for BMDCs.

Further, the obtained bone marrow cells were cultured in a DMEM including 20% heat-inactivated FBS, a 30% L929 cell culture supernatant containing a macrophage colony-stimulating factor, and 1% penicillin-streptomycin for BMDMs.

<2-1> Analysis of Inhibitory Activity of Sargassum muticum Extracts on Pro-Inflammatory Cytokine Production in CpG-ODN-Stimulated Bone Marrow-Derived Macrophage and Dendritic Cells

In order to check whether the Sargassum muticum extracts obtained in the present invention has an anti-inflammatory and an immunomodulatory activity, the following experiments were conducted with the Sargassum muticum extracts obtained in Example <1-1>. That is, bone marrow-derived macrophage and dendritic cells cultured as described above were seeded at 1×10⁵ cells/0.5 ml in a 48-well plate. Then, these cells were treated with the Sargassum muticum extracts at each concentration (0, 5, 10, 20, and 50 μg/ml) for 1 hour before stimulation with CpG-ODN (1 μM). Supernatants were harvested 18 hour after stimulation. Amounts of IL-12 p40, IL-6, and TNF-α were measured using ELISA (BD PharMingen, CA, USA and R&D system, MN, USA). At this time, a non-treated group, a group treated with the extracts only, and a group treated with CpG-ODN only without treatment with the extracts were used as a control group.

As a result, as illustrated in FIG. 2, when the bone marrow-derived macrophage (2A) and dendritic cells (2B) were stimulated with CpG-ODN only, production of the pro-inflammatory cytokines IL-12 p40, IL-6, and TNF-α remarkably increased as compared with the group which was not stimulated with CpG-ODN. In contrast, when both of the bone marrow-derived macrophage (2A) and dendritic cells (2B) were pre-treated with the Sargassum muticum extracts of the present invention, production of the pro-inflammatory cytokines was remarkably inhibited in a dose-dependent manner.

According to this result, it was confirmed that the Sargassum muticum extracts has an activity of effectively inhibiting the production of pro-inflammatory cytokines.

<2-2> Analysis of Inhibitory Activity of Sargassum muticum Extracts on Pro-Inflammatory Cytokine Production in LPS-Stimulated Bone Marrow-Derived Macrophage and Dendritic Cells

Further, in order to check whether the Sargassum muticum extracts of the present invention also inhibits the production of the pro-inflammatory cytokines stimulated by LPS, the experiments were conducted in the same manner as Example <2-1> except that instead of CpG-ODN, LPS was used at a concentration of 10 ng/ml.

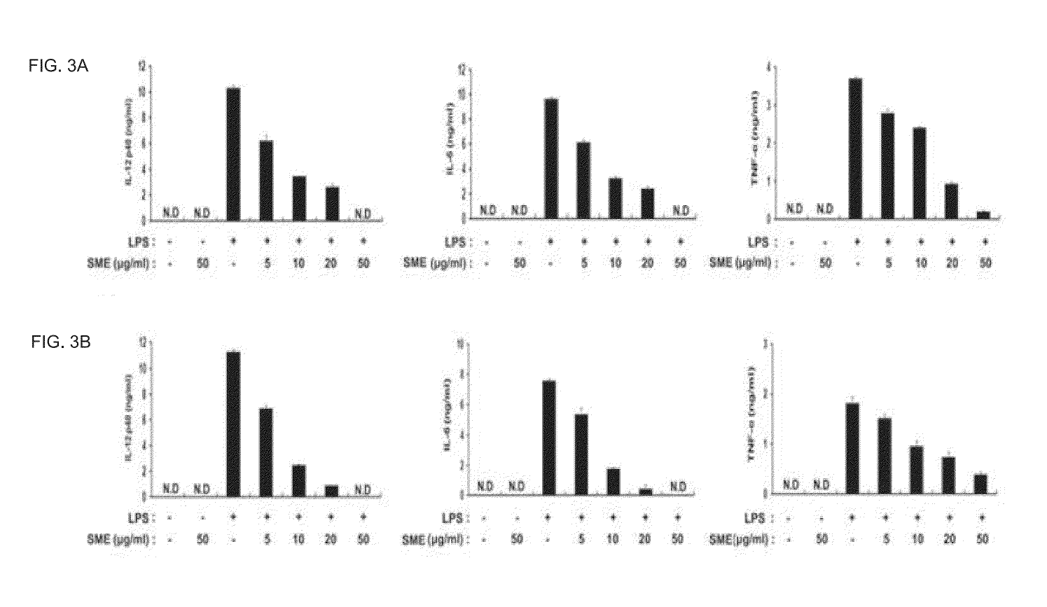

As a result, as illustrated in FIG. 3, it was confirmed that the Sargassum muticum extracts of the present invention has an activity of effectively inhibiting production of the pro-inflammatory cytokines in both of the bone marrow-derived macrophage (3A) and dendritic cells (3B) stimulated by LPS.

<2-3> Analysis of Inhibitory Activity of Apo-9′-Fucoxanthinone Compound on Pro-Inflammatory Cytokine Production in CpG-ODN-Stimulated Bone Marrow-Derived Macrophage and Dendritic Cells

In order to check whether an apo-9′-fucoxanthinone compound isolated and purified from Sargassum muticum extracts has an effect of inhibiting production of the pro-inflammatory cytokines, the experiments were conducted in the same manner as Example <2-1> except that instead of Sargassum muticum extracts, an apo-9′-fucoxanthinone compound was used at each concentration (0, 5, 10, 20, and 50 μM).

As a result, when the cells were treated with only the apo-9′-fucoxanthinone compound at a concentration of 50 μM, the pro-inflammatory cytokines was not produced.

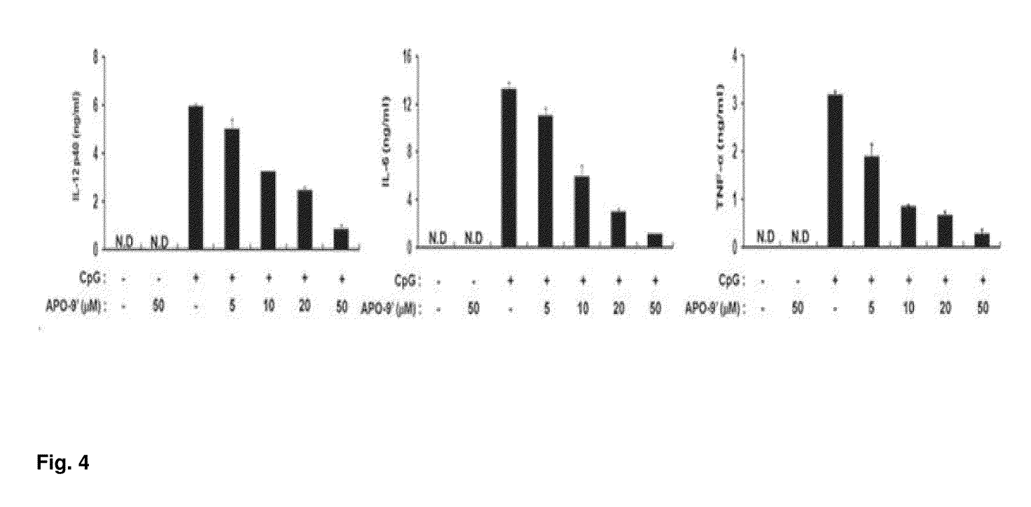

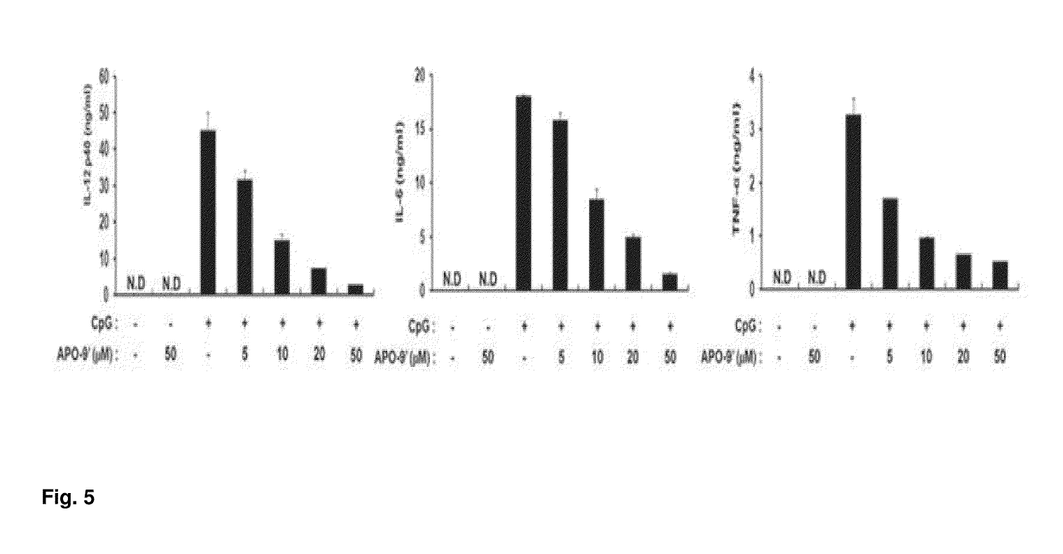

Meanwhile, it was confirmed that the apo-9′-fucoxanthinone compound has an activity of effectively inhibiting the pro-inflammatory cytokine production in both of the bone marrow-derived macrophage and dendritic cells stimulated by CpG-ODN (refer to FIG. 4 and FIG. 5).

According to this result, the inventors of the present invention found that the Sargassum muticum extracts and the apo-9′-fucoxanthinone compound of the present invention have activities of effectively inhibiting production of the pro-inflammatory cytokines stimulated by LPS and CpG-ODN, and, thus, expected that the Sargassum muticum extracts and the apo-9′-fucoxanthinone compound can prevent and treat diseases which may be caused by inflammation.

### Example 3

### Analysis of Effect of Apo-9′-Fucoxanthinone Compound on Phosphorylation of MAPK and Degradation and Phosphorylation of IκBα

In order to check whether the apo-9′-fucoxanthinone compound has an effect on activities of MAPK and NF-κB that are upstream signal transduction pathways for pro-inflammatory cytokine production, a Western blot analysis was conducted as follows. That is, the macrophages were dispensed to 60-mm culture dishes at 4×10⁶ cells per dish, and then cultured for 24 hours at 37° C. The cells were pre-treated with the apo-9′-fucoxanthinone compound at a concentration of 20 μM for 1 hour before treatment with CpG-ODN. The cells were collected and dissolved using a lysis buffer (PRO-PREP Lysis buffer, iNtRON Biotechnology). Protein samples were electrophoresed in 10%-SDS PAGE, and then electrophoresed in a polyvinylidene fluoride membrane, and then treated and reacted with phospho-p44/42 (P-ERK p44/p42), phospho-p38 (P-p38), p38 MAPK, phospho-SAPK/JNK (P-JNK), phospho-IκBα, and IκBα (Cell Signaling Technology, MA, USA) as 1/1000 diluted primary antibodies. Thereafter, the membrane was washed and treated and reacted with horseradish peroxidase-linked anti-rabbit IgG (Cell Signaling Technology). Then, a signal was checked using a WEST-ZOL plus western blot detection system (iNtRON Biotechnology).

According to the result of the Western blot analysis, as illustrated in FIG. 6, phosphorylation of ERK1/2 (ERK p44/p42) was strongly inhibited with the treatment of the apo-9′-fucoxanthinone compound (refer to FIG. 6A), but the apo-9′-fucoxanthinone compound did not greatly affect degradation and phosphorylation of IκBα (refer to FIG. 6B). According to this result, the inventors of the present invention confirmed that since the apo-9′-fucoxanthinone compound inhibited an activity of ERK1/2 MAPK of the MAPK family via the inhibition of phosphorylation thereof but did not affect phosphorylation and degradation of IκBα, the apo-9′-fucoxanthinone compound cannot inhibit an activity of NF-κB. Therefore, according to this result, the inventors of the present invention confirmed that the apo-9′-fucoxanthinone compound inhibits production of the pro-inflammatory cytokines in the CpG-ODN-stimulated macrophage via inhibition of phosphorylation of ERK1/2 MAPK.

### Example 4

### Analysis on Inhibition of AP-1 Reporter Activity by Apo-9′-Fucoxanthinone Compound

It is known that activation of an MAPK signaling pathway activates a transcriptional factor AP-1, thereby increasing a DNA binding activity of AP-1 and expression of an AP-1-dependent gene. Thus, in order to check whether the apo-9′-fucoxanthinone compound of the present invention inhibits a transcriptional activity of AP-1 induced by stimuli of CpG-ODN, the inventors of the present invention checked inhibition of an AP-1 reporter activity by luciferase assay. Firstly, HEK239T cells were plated in a 24-well plate and cultured overnight. Then, HEK293T cells were transfected with pcDNA3 (empty vector) or pcDNA3-mTLR9 that expresses murine TLR9, with an AP-1 reporter gene and pRLnull (Promega, WI, USA) using a Fugene 6 (Roche, UN, USA). Cells were then further incubated for 24 hour, and then pre-treated with the apo-9′-fucoxanthinone compound at each concentration as described in the above Examples for 1 hour before stimulation with CpG DNA (1 μM). After further incubation for 18 hour, the cells were lysed with a passive lysis buffer (Promega), and then analyzed using a dual luciferase reporter assay system (Promega). All of the experiments were repeated three times, and the results thereof were expressed in the fold increase.

According to the assay result, as illustrated in FIG. 7, when the pcDNA3 plasmid was transfected into the cells without treatment with the apo-9′-fucoxanthinone compound, an AP-1-dependent luciferase activity induced by stimuli of CpG-ODN rarely occurred, whereas in the group in which the murine TLR9-expressing plasmid was transfected into the cells, an AP-1-dependent luciferase activity induced by stimuli of CpG-ODN increased by about 8,000 times.

However, as for the groups treated with the apo-9′-fucoxanthinone compound before stimulation with CpG-ODN, when the murine TLR9-expressing plasmid was transfected into the cells, an AP-1-dependent luciferase activity decreased in a dose-dependent manner of the apo-9′-fucoxanthinone compound.

### Example 5

### Analysis on Effect of Apo-9′-Fucoxanthinone Compound on NF-κB Reporter Activity

When NF-κB is activated as a transcriptional factor, it translocates into the nucleus and binds to a promoter site to enable target genes to be transcribed. Thus, the inventors of the present invention checked whether the apo-9′-fucoxanthinone compound inhibits an NF-κB reporter activity induced by stimuli of CpG-ODN by luciferase assay. The experiments were conducted in the same manner as Example 4 except that instead of an AP-1 reporter gene, an NF-κB reporter gene was used.

According to the assay result, as illustrated in FIG. 8, when the pcDNA3 plasmid was transfected into the cells without treatment with the apo-9′-fucoxanthinone compound, an NF-κB-dependent luciferase activity induced by stimuli of CpG-ODN rarely occurred.

However, it was exhibited that when the group in which pcDNA3-mTLR9 was transfected into the cells without pre-treatment with the apo-9′-fucoxanthinone compound, a transcriptional activity of NF-κB sharply increased. However, it was exhibited that when these cells were pre-treated with the apo-9′-fucoxanthinone compound at each concentration, a transcriptional activity of NF-κB was not inhibited significantly as compared with inhibition of a transcriptional activity of AP-1.

According to this result, the inventors of the present invention confirmed that the apo-9′-fucoxanthinone compound of the present invention can inhibit TLR9-dependent AP-1 transcriptional activity induced by stimuli of CpG-ODN with considerable effect, but cannot inhibit NF-κB transcriptional activity effectively.

### Example 6

### Effect of Apo-9′-Fucoxanthinone Compound on Secretion of IL-1β Induced by LPS and ATP, and on Pyroptosis of Macrophages

When LPS primed bone marrow-derived macrophages are treated with ATP, NLPR3 inflammasome is activated, and, thus, IL-1β is secreted. Therefore, in order to check whether the apo-9′-fucoxanthinone compound has an effect of inhibiting the NLRP3 inflammasome, the inventors of the present invention checked a degree of IL-1β production induced by ATP in LPS primed BMDMs using ELISA To do so, the bone marrow-derived macrophages cultured in the same manner as Example 2 were dispensed at 1×10⁵ cells/0.5 ml in a 48-well plate, and these cells were treated with LPS at a concentration of 10 ng/ml for 18 hours. Then, the cells were treated with the apo-9′-fucoxanthinone compound at each concentration (0, 6.25, 12.5, 25, and 50 μM) for 1 hour. At 2 hours after treatment with ATP at a concentration of 5 mM, a cell culture medium was obtained and an expression amount of IL-1β was measured using ELISA (R&D system, MN, USA). Further, as a positive control group, a group treated with 10 μM parthenolide (described as “part” in the drawings) known as an NLRP3 inflammasome inhibitor was used.

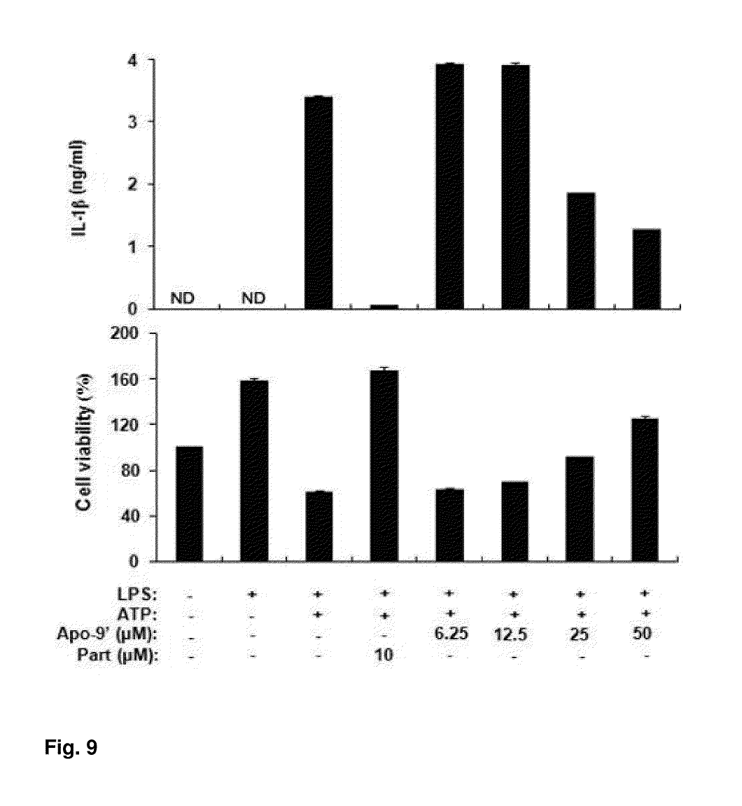

According to the assay result, as illustrated in the upper graph in FIG. 9, LPS treatment alone showed no production of IL-1β while LPS primed BMDMs treated with ATP induced significantly increased production of IL-1β. Further, when LPS primed BMDMs were treated with the apo-9′-fucoxanthinone of the present invention, production of IL-1β was effectively inhibited in a dose-dependent manner.

Meanwhile, according to the conventional reports, when LPS primed bone marrow-derived macrophages are treated with ATP, NLRP3 inflammasome-dependent pyroptotic cell death is induced. Therefore, in order to analyze whether the apo-9′-fucoxanthinone compound of the present invention has an effect of inhibiting such pyroptotic cell death, the inventors of the present invention conducted an MTT assay to measure cell viability.

That is, the cells treated as described above were treated with 0.2 mg of an MTT reagent and cultured at a temperature of 37° C. for 4 hours. After culture, the cells were collected and formazan crystals in each well were dissolved in 250 μl of DMSO, and then, an absorbance at 540 nm was measured to measure cell viability.

According to the result thereof, as illustrated in the lower graph in FIG. 9, it was exhibited that the apo-9′-fucoxanthinone compound has an effect of inhibiting pyroptotic cell death of the bone marrow-derived macrophages treated with both of LPS and ATP in a dose-dependent manner.

According to this result, the inventors of the present invention confirmed that the apo-9′-fucoxanthinone compound can inhibit an activity of NLRP3 inflammasome and protect the bone marrow-derived macrophages frompyroptotic cell death. e the present invention has been exhibited and described with reference to preferable Examples thereof, it will be understood by those skilled in the art that various changes in form and details may be made therein without departing from the spirit and scope of the present invention as defined by the appended claims. Therefore, the disclosed Examples should not be considered in view of explanation, but no limitation. The technical scope of the present invention is taught in the claims, but not the detailed description and all the differences in the equivalent scope thereof should be construed as falling within the present invention.

1. A method for preventing and treating inflammatory diseases or immune diseases comprising administering to a patient in need thereof an effective amount of an apo-9′-fucoxanthinone expressed by the following chemical formula 1;

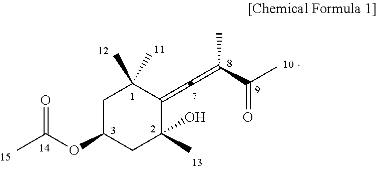

2. The method of claim 1, wherein the apo-9′-fucoxanthinone is isolated from Sargassum muticum extracts.

3. The method of claim 1, wherein the apo-9′-fucoxanthinone has an inhibitory effect on production of pro-inflammatory cytokines stimulated with LPS (lipopolysaccharide) or CpG-ODN (CpG-oligodeoxynucleotide).

4. The method of claim 3, wherein the pro-inflammatory cytokines are IL-12, IL-6, or TNF-α.

5. The method of claim 1, wherein the apo-9′-fucoxanthinone inhibits activities of MAPK (MAP Kinase) and AP-1.

6. The method of claim 1, wherein the apo-9′-fucoxanthinone has an activity of inhibiting NLRP3 (the nucleotide-binding oligomerization domain (NOD)-like receptor family, pyrin domain containing 3) inflammasome.

7. The method of claim 2, wherein the Sargassum muticum extracts are extracted with an extractant selected from the group consisting of water, C1 to C4 alcohol, hexane, ethylacetate, butylene glycol, propylene glycol, glycerin, ether, chloroform, methylene chloride, n-butanol, and mixed solvents thereof.

8. The method of claim 1, wherein the apo-9′-fucoxanthinone is included at a concentration of 5 to 50 μM in the composition.

9. The method of claim 1, wherein the inflammatory diseases include sepsis exhibiting a systemic inflammatory response caused by infection with microorganisms or endotoxic shock.

10. The method of claim 1, wherein the immune diseases include one or more diseases selected from the group consisting of alopecia greata, dermatitis, allergy, atopy, asthma, conjunctivitis, periodontitis, rhinitis, tympanitis, pharyngolaryngitis, tonsillitis, pneumonia, gastric ulcer, gastritis, Crohn's disease, colitis, hemorrhoids, ankylosing spondylitis, lupus, fibromyalgia, psoriasis, arthritis, ostarthritis, rheumatoid arthritis, shoulder joint diseases, tendinitis, tenosynovitis, peritendinitis, myositis, hepatitis, cystitis, nephritis, sjogren's syndrome, multiple sclerosis, inflammatory bowel disease, type 1 diabetes, scleroderma, gout, neurodegenerative diseases, type 2 diabetes, silicosis, atherosclerosis, autoimmune inflammatory diseases, chronic kidney diseases and genetically inherited periodic fever syndromes, CAPS (Cryopyrin-associated periodic syndrome), FACS (familial cold autoinflammatory syndrome), MWS (Muckle-Wells syndrome), NOMID (neonatal onset multisystem inflammatory disease), and acute and chronic inflammatory diseases.

11. A functional food comprising apo-9′-fucoxanthinone expressed by the following chemical formula 1 or Sargassum muticum extracts for preventing and treating inflammatory diseases or immune diseases;

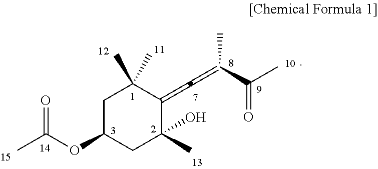

12. A pharmaceutical composition comprising Sargassum muticum extracts as an effective component for preventing and treating inflammatory diseases or immune diseases.

13. The pharmaceutical composition of claim 12, wherein the Sargassum muticum extracts are extracted with an extractant selected from the group consisting of water, C1 to C4 alcohol, hexane, ethylacetate, butylene glycol, propylene glycol, glycerin, ether, chloroform, methylene chloride, n-butanol, and mixed solvents thereof.

14. The pharmaceutical composition of claim 12, wherein the Sargassum muticum extracts are included at a concentration of 5 to 50 μg/ml in the composition.

15. The pharmaceutical composition of claim 12, wherein the Sargassum muticum extracts includes an apo-9′-fucoxanthinone compound.
